(19) United States
(12) Patent Application Publication
(10) Pub. No.: US 2026/0285810 A1
(43) Pub. Date: Sep. 24, 2026

(54) CRYSTAL FORM OF MUSCARINE ANTAGONIST, METHOD FOR PREPARING SAME, AND USE THEREOF

(71) Applicant: SICHUAN KELUN PHARMACEUTICAL RESEARCH INSTITUTE CO., LTD., Chengdu (CN)

(72) Inventors: Jiao ZHANG, Chengdu (CN); Xiaobo MA, Chengdu (CN); Tianming WANG, Chengdu (CN); Gao WANG, Chengdu (CN); Wenhao LI, Chengdu (CN); Zhou SHI, Chengdu (CN); Junzhu PAN, Chengdu (CN); Chengxi YANG, Chengdu (CN); Dong ZHAO, Chengdu (CN); Sichuan LIU, Chengdu (CN)

(21) Appl. No.: 19/474,114

(22) PCT Filed: Apr. 30, 2024
(86) PCT No.: PCT/CN2024/090925

§ 371 Date: Oct. 9, 2025

(30) Foreign Application Priority Data
May 9, 2023 (CN) 202310515719.8
Jan. 23, 2024 (CN) 202410096775.7

(51) Int. Cl. C07D 211/62; A61K 31/4545; A61K 45/06

(52) U.S. Cl. CPC C07D 211/62; A61K 31/4545; A61K 45/06

(57) ABSTRACT

The present application discloses a crystal form of a muscarine antagonist, a method for preparing same, and use thereof, and particularly, a crystal form A of revefenacin. The crystal form A of revefenacin comprises an X-ray powder diffraction pattern having characteristic peaks at diffraction angles 2θ of 4.7±0.2, 9.2±0.2, 12.7±0.2, 18.2±0.2, 18.5±0.2, 20.7±0.2, 21.3±0.2, and 25.6±0.2. The crystal form of the present disclosure features a regular sheet shape, a high crystallinity, a high dissolution rate, and high fluidity and bulk density.

### CROSS-REFERENCE TO RELATED APPLICATIONS

The present application is a national stage application of International Patent Application No. PCT/CN2024/090925, filed on Apr. 30, 2024, entitled: “CRYSTAL FORM OF MUSCARINE ANTAGONIST, METHOD FOR PREPARING SAME, AND USE THEREOF,” which claims the benefit of and priority to Chinese Patent Application No. 202310515719.8, filed on May 9, 2023, and Chinese Patent Application No. 202410096775.7, filed Jan. 23, 2024, each of which is incorporated herein by reference.

### TECHNICAL FIELD

The present disclosure belongs to the field of pharmaceutical crystal forms, and relates to a crystal form of a muscarine antagonist, a method for preparing same, and the use thereof, in particular to a crystal form of revefenacin and a method for preparing same.

### BACKGROUND ART

Revefenacin, whose chemical name is 1-(2-(4-(((4-carbamoylpiperidin-1-yl)methyl)-N-methylbenzamido)ethyl) piperidin-4-yl[1,1′-biphenyl]-2-ylcarbamate, has the following structural formula:

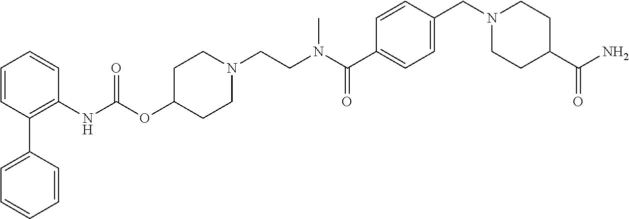 [SMILES: CN(CCN1CCC(OC(=O)Nc2ccccc2-c2ccccc2)CC1)C(=O)c1ccc(CN2CCC(C(N)=O)CC2)cc1]

Revefenacin is a novel biphenyl compound with an activity of a muscarine receptor antagonist or an anticholinergic activity, which prevents acetylcholine-induced bronchoconstriction by blocking M3 receptors in airway smooth muscle, thereby exerting a bronchodilatory effect. In November 2018, the U.S. Food and Drug Administration (FDA) approved the inhalation solution of revefenacin for maintenance treatment in patients with chronic obstructive pulmonary disease (COPD).

There are many reports on solid forms of revefenacin. U.S. patent application US 2007/0112027 discloses crystal forms I and II of the free base hydrate of revefenacin. Chinese patent application CN 102470130 A discloses two crystal forms, form Ill and form IV, of the free base anhydrate of revefenacin. Among the crystal forms of revefenacin, form I, form Il and form IV require harsh crystallization conditions, and are not suitable for industrial production and process scale-up. Moreover, the form I crystal exhibits an agglomerated crystal morphology (see patent application CN 101163677 A, FIG. 22), which complicates impurity removal during production and results in poor process robustness. For form III, crystallization from acetonitrile involves a complex process with a low yield (75%), while the yield in crystallization from toluene can be increased to 90% or more; however, toluene is a class 2 solvent, and the use of toluene as a crystallization solvent may result in excessive residual solvent levels. For solution-based inhalation preparations, it is necessary to develop new crystal forms that are relatively stable, have a simple crystallization process easy for industrial scale-up production, and exhibit low hygroscopicity and moisture absorption.

### SUMMARY OF THE INVENTION

A first aspect of the present disclosure provides a crystal form A of revefenacin, which is a free base form containing water of crystallization and having a water content of 8.0 wt %-10 wt % within a humidity range of 30%-90%; when stored under a humidity condition of 80%+2% for 24 hours, the crystal form exhibits a hygroscopic weight gain of <2%; the crystal form features a regular sheet shape and a high crystallinity, and has a high dissolution rate during the preparation of an inhalation solution.

The crystal form A of revefenacin of the present disclosure comprises an X-ray powder diffraction pattern having characteristic peaks at diffraction angles 2θ of 4.7±0.2°, 9.2±0.2°, 12.7±0.2°, 18.2±0.2°, 18.5±0.2°, 20.7±0.2°, 21.3±0.2° and 25.6±0.2°.

In some embodiments of the present disclosure, the crystal form A of revefenacin comprises an X-ray powder diffraction pattern further having at least one characteristic peak at diffraction angles 2θ of 13.3±0.2°, 13.7±0.2°, 14.0±0.2°, 16.1±0.2°, 16.7±0.2°, 17.4±0.2°, 18.8±0.2°, 19.4±0.2°, 22.1±0.2°, 22.3±0.2°, 23.0±0.2°, 24.2±0.2° and 33.5±0.2°, such as 1, 2, 3, 4, 5, 6, 7, 8, 9, 10, 11, 12 or 13 characteristic peaks.

In some embodiments of the present disclosure, the crystal form A of revefenacin comprises an X-ray powder diffraction pattern having characteristic peaks at diffraction angles 2θ of 4.7±0.2°, 9.2±0.2°, 12.7±0.2°, 13.3±0.2°, 13.7±0.2°, 14.0±0.2°, 16.1±0.2°, 16.7±0.2°, 17.4±0.2°, 18.2±0.2°, 18.5±0.2°, 18.8±0.2°, 19.4±0.2°, 20.7±0.2°, 21.3±0.2°, 22.1±0.2°, 22.3±0.2°, 23.0±0.2°, 24.2±0.2°, 25.6±0.2° and 33.5±0.2°.

In some embodiments of the present disclosure, the crystal form A of revefenacin comprises an X-ray powder diffraction pattern further having at least one characteristic peak at diffraction angles 2θ of 8.9±0.2°, 9.6±0.2°, 9.8±0.2°, 20.1±0.2°, 23.3±0.2°, 26.4±0.2°, 26.9±0.2°, 27.6±0.2°, 28.5±0.2°, 29.1±0.2°, 29.4±0.2°, 30.1±0.2° and 32.9±0.2°, such as 1, 2, 3, 4, 5, 6, 7, 8, 9, 10, 11, 12 or 13 characteristic peaks.

In some embodiments of the present disclosure, the crystal form A of revefenacin comprises an X-ray powder diffraction pattern having characteristic peaks at diffraction angles 2θ of 4.7±0.2°, 8.9±0.2°, 9.2±0.2°, 9.6±0.2°, 9.8±0.2°, 12.7±0.2°, 13.3±0.2°, 13.7±0.2°, 14.0±0.2°, 16.1±0.2°, 16.7±0.2°, 17.4±0.2°, 18.2±0.2°, 18.5±0.2°, 18.8±0.2°, 19.4±0.2°, 20.1±0.2°, 20.7±0.2°, 21.3±0.2°, 22.1±0.2°, 22.3±0.2°, 23.0±0.2°, 23.3±0.2°, 24.2±0.2°, 25.6±0.2°, 26.4±0.2°, 26.9±0.2°, 27.6±0.2°, 28.5±0.2°, 29.1±0.2°, 29.4±0.2°, 30.1±0.2°, 32.9±0.2° and 33.5±0.2°.

In some embodiments of the present disclosure, the crystal form A of revefenacin comprises an X-ray powder diffraction pattern substantially as shown in FIG. 1.

In some embodiments of the present disclosure, the crystal form A of revefenacin comprises an X-ray powder diffraction pattern as shown in FIG. 1.

In some embodiments of the present disclosure, the crystal form A of revefenacin is a hydrate crystal form containing water of crystallization and having a water content of 8.0 wt %-10 wt % as determined by Karl Fischer titration.

In some embodiments of the present disclosure, the crystal form A of revefenacin is a revefenacin trihydrate.

In some embodiments of the present disclosure, the crystal form A of revefenacin has an endothermic peak between 85° C. and 125° C., and the endothermic peak has an onset temperature of 88.3° C.±5° C.

In some embodiments of the present disclosure, the crystal form A of revefenacin comprises a DSC curve substantially as shown in FIG. 2.

In some embodiments of the present disclosure, the crystal form A of revefenacin comprises a DSC curve as shown in FIG. 2.

In some embodiments of the present disclosure, the crystal form A of revefenacin shows a weight loss of 9.2%±0.2% in a temperature range of 35° C.-100° C.

In some embodiments of the present disclosure, the crystal form A of revefenacin comprises a TGA curve substantially as shown in FIG. 3.

In some embodiments of the present disclosure, the crystal form A of revefenacin comprises a TGA curve as shown in FIG. 3.

In some embodiments of the present disclosure, the crystal form A of revefenacin features a regular sheet shape.

In some embodiments of the present disclosure, the crystal form A of revefenacin comprises a polarized light microscopy (PLM) image as shown in FIG. 4.

A second aspect of the present disclosure provides a revefenacin hydrate single crystal, which belongs to the triclinic crystal system and has a space group of P-1 with crystal axes a=9.5522 (4) Å, b=9.9096 (4) Å, and c=18.3690 (9) Å, interaxial angles α=86.419 (4)°, β=86.906 (4)′, and γ=89.544 (3)°, a Z value of 2 for the number of asymmetric units per unit cell, and each unit cell containing one revefenacin molecule and three water molecules.

A third aspect of the present disclosure provides a method for preparing a crystal form A of revefenacin, comprising the following steps:
step I: mixing revefenacin with a dissolution solvent, stirring and heating the resulting mixture to give a clear solution, wherein the dissolution solvent is a mixed solvent of ethanol/water or a mixed solvent of acetone/water, for example, the dissolution solvent is a mixed solvent of ethanol/water;
step II: cooling the solution to 35° C.-45° C., stirring the solution while maintaining the temperature for crystal growth to obtain a revefenacin suspension solution;
step III: under a condition of 35° C.-45° C., adding a precipitation solvent dropwise to the suspension solution obtained in step II; and step IV: cooling the suspension solution to 0° C.-25° C. for crystal growth, and then performing filtration and drying to a constant weight to obtain the crystal form A of revefenacin.

In some embodiments of the present disclosure, in the preparation method described above, in step I, the mass-to-volume ratio of revefenacin to the dissolution solvent, expressed in g/mL, is 1:4-5.

In some embodiments of the present disclosure, in the preparation method described above, in step I, revefenacin is added to the dissolution solvent, stirred, and heated to 50° C.-55° C. to give a clear solution.

In some embodiments of the present disclosure, in the preparation method described above, the dissolution solvent is a mixed solvent of ethanol/water, preferably, the dissolution solvent is a mixed solvent of ethanol and water in a volume ratio of 1-2:1, preferably a volume ratio of 2:1 or 1:1.

In some embodiments of the present disclosure, in the preparation method described above, the dissolution solvent is a mixed solvent of acetone/water, preferably, the dissolution solvent is a mixed solvent of acetone and water in a volume ratio of 1-4:1, preferably a volume ratio of 2-4:1, such as a volume ratio of 4:1, 3:1 or 2:1.

In some embodiments of the present disclosure, in the preparation method described above, in step II, seed crystals of the crystal form A of revefenacin are added.

In some embodiments of the present disclosure, in the preparation method described above, in step II, the seed crystals are added in an amount of 1 wt % to 2 wt %, preferably 1 wt %, of the revefenacin.

In some embodiments of the present disclosure, in the preparation method described above, in step II, stirring while maintaining the temperature for crystal growth is performed over 1-4 h, preferably 2 h; the stirring rate is 100-800 rpm, preferably 400-600 rpm.

In some embodiments of the present disclosure, in the preparation method described above, in step III, the precipitation solvent is a mixed solvent of ethanol and water, preferably a mixed solvent of ethanol and water in a volume ratio of 1:3-5, such as a mixed solvent of ethanol and water in a volume ratio of 1:3, 1:4 or 1:5.

In some embodiments of the present disclosure, in the preparation method described above, in step III, the precipitation solvent is a mixed solvent of acetone/water, preferably a mixed solvent of acetone and water in a volume ratio of 1:3-5, such as a mixed solvent of acetone and water in a volume ratio of 1:3, 1:4 or 1:5.

In some embodiments of the present disclosure, in the preparation method described above, the dissolution solvent is a mixed solvent of ethanol/water, preferably a mixed solvent of ethanol and water in a volume ratio of 1-2:1, such as a mixed solvent of ethanol and water in a volume ratio of 2:1 or 1:1;

in step III, the precipitation solvent is a mixed solvent of ethanol and water, preferably a mixed solvent of ethanol and water in a volume ratio of 1:3-5, such as a mixed solvent of ethanol and water in a volume ratio of 1:3, 1:4 or 1:5.

In some embodiments of the present disclosure, in the preparation method described above, the dissolution solvent is a mixed solvent of acetone/water, preferably a mixed solvent of acetone and water in a volume ratio of 1-4:1, preferably a volume ratio of 2-4:1, such as a volume ratio of 4:1, 3:1 or 2:1;

in step III, the precipitation solvent is a mixed solvent of acetone/water, preferably a mixed solvent of acetone and water in a volume ratio of 1:3-5, such as a mixed solvent of acetone and water in a volume ratio of 1:3, 1:4 or 1:5.

In some embodiments of the present disclosure, in the preparation method described above, the mass-to-volume ratio of revefenacin to the precipitation solvent in step III, expressed in g/mL, is 1:3-6, preferably 1:4.

In some embodiments of the present disclosure, in the preparation method described above, in step III, the dropwise addition rate of the precipitation solvent is non-linear dropwise addition, with the dropwise addition rate being slow first and then fast; preferably, the dropwise addition is performed over 2-6 h; more preferably, the dropwise addition is performed over 3-4 h.

In some embodiments of the present disclosure, in the preparation method described above, in step IV, the crystal growth is maintained for 1-10 h.

In some embodiments of the present disclosure, in the preparation method described above, in step IV, the cooling rate is 0.1° C./min-2° C./min, preferably 0.5° C./min.

In some embodiments of the present disclosure, in the preparation method described above, in step IV, drying is performed at a temperature of 25° C.-45° C. and a vacuum degree of 0.08 Mpa-0.1 Mpa for 2-20 h.

A fourth aspect of the present disclosure provides a method for preparing a revefenacin hydrate single crystal, comprising the following steps:
step I: adding an excess amount of revefenacin to an acetone/water solution, mixing uniformly, and filtering the resulting mixture to obtain a saturated solution of revefenacin; and
step II: diluting the saturated solution of revefenacin with an acetone/water solution, and leaving the solution to stand for evaporation to obtain the revefenacin hydrate single crystal.

In some embodiments of the present disclosure, in the preparation method described above, in steps I and II, the acetone/water solution has a volume ratio of acetone to water of 5-7:1, such as 5:1, 6:1 or 7:1.

In some embodiments of the present disclosure, in the preparation method described above, in step I, filtering is performed via a syringe filter, preferably a 0.25 μm syringe filter.

In some embodiments of the present disclosure, in the preparation method described above, in step II, the volume ratio of the saturated solution of revefenacin to the acetone/water solution is 1:8-10, preferably 1:9.

A fifth aspect of the present disclosure provides a pharmaceutical composition comprising the crystal form A of revefenacin according to the first aspect of the present disclosure or the revefenacin hydrate single crystal according to the second aspect of the present disclosure, and one or more pharmaceutically acceptable carriers.

In some embodiments of the present disclosure, the pharmaceutical composition optionally comprises one or more “other therapeutic agents”, wherein the “other therapeutic agents” refer to pharmacologically active substances other than revefenacin, such as other muscarine receptor antagonists or anticholinergic activity capable of exerting synergistic therapeutic effects with revefenacin.

The pharmaceutically acceptable carrier may be selected from the following ingredients: fillers (diluents), binders, wetting agents, disintegrants or excipients, etc. The unit dose of the pharmaceutical composition may be expressed as tablets, capsules, cachets, vials, powders, granules, emulsions, lozenges, suppositories, reconstitutable powders or liquid preparations, etc. Depending on the method of administration, the composition may comprise 0.1 wt % to 99 wt % of the active compound. In some embodiments, the unit dose of the pharmaceutical composition is 0.001 mg-1000 mg.

A sixth aspect of the present disclosure provides the use of the crystal form A of revefenacin according to the first aspect of the present disclosure, the revefenacin hydrate single crystal according to the second aspect of the present disclosure or the pharmaceutical composition according to the fifth aspect of the present disclosure in the manufacture of a medicament for treating chronic obstructive pulmonary disease.

A seventh aspect of the present disclosure provides the crystal form A of revefenacin according to the first aspect of the present disclosure, the revefenacin hydrate single crystal according to the second aspect of the present disclosure or the pharmaceutical composition according to the fifth aspect of the present disclosure, for use in the treatment of chronic obstructive pulmonary disease.

An eighth aspect of the present disclosure provides a method for treating chronic obstructive pulmonary disease, comprising administering to a subject in need thereof a therapeutically effective amount of the crystal form A of revefenacin according to the first aspect of the present disclosure, the revefenacin hydrate single crystal according to the second aspect of the present disclosure or the pharmaceutical composition according to the fifth aspect of the present disclosure.

The present disclosure has the following beneficial effects:

The crystal form of the present disclosure features a regular sheet shape, a high crystallinity, a large particle size, and superior powder properties, with a high dissolution rate and high fluidity and bulk density; and the crystal form is easy to pulverize, low in cost, and more conducive to implementation on a commercial industrial scale.

The preparation method of the present disclosure features simple and efficient operation, which can effectively avoid oiling out during crystallization, consumes less time and energy, and delivers a product with a purity exceeding 99.5% and a yield of ≥90.0%; and the process demonstrates excellent robustness and good repeatability, ensuring consistent crystal forms between batches.

### BRIEF DESCRIPTION OF THE DRAWINGS

FIG. 1 shows an X-ray powder diffraction (XRPD) pattern of crystal form A of revefenacin.

FIG. 2 shows a DSC pattern of crystal form A of revefenacin.

FIG. 3 shows a TGA pattern of crystal form A of revefenacin.

FIG. 4 shows a polarized light microscopy (PLM) image (50× magnification) of crystal form A of revefenacin.

FIG. 5 shows a dynamic vapor sorption (DVS) pattern of crystal form A of revefenacin.

FIG. 6 shows XRPD patterns of revefenacin at various water contents, where curves from bottom to top represent XRPD patterns of revefenacin with KF water contents of 17 wt %, 8.9 wt %, 8.3 wt %, and 7.1 wt %, respectively.

FIG. 7 shows XRPD patterns of crystal form A of revefenacin during temperature- and humidity-mediated crystal transformation.

FIG. 8 shows XRPD patterns of crystal form A of revefenacin after accelerated and long-term storage.

FIG. 9 shows a single crystal structure diagram of revefenacin hydrate single crystal.

FIG. 10 shows a simulated XRD pattern calculated from revefenacin single crystal.

FIG. 11 shows a particle size distribution profile of crystal form A of revefenacin.

### DETAILED DESCRIPTION OF EMBODIMENTS

The present disclosure is further illustrated by the following examples, which are merely illustrative of the technical solutions of the present disclosure and are not intended to limit the scope of the present disclosure. Those skilled in the art can make some non-essential improvements and adjustments, which still fall within the scope of protection of the present disclosure.

### Definitions

Unless otherwise indicated, all technical terms and scientific terms used herein have the same meanings as commonly understood by those skilled in the art to which the present disclosure belongs. In case of a contradiction, the definitions herein shall prevail. When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value. Where a range of numerical values is recited herein, unless otherwise stated, the range is intended to include the endpoints thereof, and all integers and fractions (decimal values) within the range.

The term “about” when used in conjunction with a numerical variable generally means that the value of the variable and all values of the variable are within experimental error (e.g., within the 95% confidence interval for the mean) or within ±20%, ±10%, ±5% or ±2% of the specified value.

The term “comprising” or similar expressions with the same meanings “including”, “containing”, “having”, etc. are open-ended and do not exclude additional unlisted elements, steps or ingredients. The expression “consisting of” exclude any unspecified element, step or ingredient. The term “consisting essentially of” means that the range is limited to the specified elements, steps or ingredients plus optional elements, steps or ingredients that do not substantively affect the basic and new features of the claimed subject matter. It should be understood that the term “comprising” and similar terms encompass the terms “consisting essentially of” and “consisting of”.

The terms “optional” or “optionally” used herein mean that the subsequently described event or circumstance may or may not occur, and the description includes the occurrence and the non-occurrence of the event or circumstance.

Unless otherwise indicated, percentages, parts, etc. herein are calculated by weight.

As used herein, the term “crystal form” or “crystal” refers to any solid substance that exhibits a three-dimensional ordering that, in contrast to amorphous solid substances, produces a characteristic XRPD pattern with well-defined peaks.

As used herein, the term “seed crystal” refers to an additive that can form crystal nuclei in a crystallization method to accelerate or promote the growth of crystals of enantiomers having the same crystal form or stereochemical configuration as the seed crystal.

As used herein, the term “X-ray powder diffraction pattern” or “XRPD pattern” refers to an experimentally observed diffraction pattern or parameters, data or values derived therefrom. XRPD patterns are typically characterized by peak positions (abscissa) and/or peak intensities (ordinate).

As used herein, the term “diffraction angle” or “2θ” refers to a peak position expressed in degrees (′) based on the experimental setup of the X-ray diffraction experiment, and is usually an abscissa unit in a diffraction pattern. The experimental setup requires that if a reflection is diffracted when the incoming beam forms an angle θ with a certain lattice plane, the reflected beam is recorded at an angle 2θ. It should be understood that reference herein to specific 2θ values for a specific crystal forms are intended to mean the 2θ values (expressed in degrees) measured using the X-ray diffraction experimental conditions described herein. For example, as described herein, monochromatic radiation of Cu-Kα (Kα1 (Å): 1.5406) is used. The XRPD patterns herein are preferably collected on a PANalytacal X'Pert3 Powder X-ray powder diffraction analyzer, and the transmission mode is preferably collected on a PANalytacal X'Pert3 Powder X-ray powder diffraction analyzer.

As used herein, the terms “substantially the same” or “substantially as shown in FIG. x” with respect to X-ray diffraction peaks mean that representative peak positions and intensity variations are taken into account. For example, those skilled in the art will appreciate that peak positions (20) may show some variation, typically up to 0.1 to 0.2 degrees, and that the instrument used to measure diffraction may also cause some variation. In addition, those skilled in the art will appreciate that relative peak intensities may vary depending on instrument-to-instrument differences as well as degree of crystallinity, preferred orientation, prepared sample surfaces, and other factors known to those skilled in the art.

Similarly, as used herein, the expression “substantially as shown in FIG. x” with respect to DSC patterns and TGA patterns is also intended to encompass variations known to those skilled in the art relating to these analytical techniques. For example, well-defined peaks in DSC patterns typically have variations of up to ±5° C., and broad peaks have even greater variations (e.g., up to ±10° C.).

As used herein, the term “room temperature” refers to 20° C.±5° C.

### Characterization of the Crystal Form

The crystal forms prepared in the examples are characterized by X-ray powder diffraction (XRPD), differential scanning calorimetry (DSC), thermogravimetric analysis (TGA), polarized light microscopy (PLM) and dynamic vapor sorption (DVS).

### (1) X-Ray Powder Diffraction (XRPD):

The XRPD pattern of the crystal form is collected using an X'Pert3 Powder diffractometer, which uses Cu palladium irradiation and performs detection using Absolute scan at room temperature. The detection range is from 3.5° to 40°, with a step size of 0.013°, a dwell time of 50 s, and one scan.

### (2) DSC

The DSC pattern of the crystal form is collected using a TA DSC2500 differential scanning calorimeter. The test temperature range is from 35° C. to 150° C., with a heating rate of 10° C./min. During the test, purging is performed with nitrogen at a flow rate of 50 mL/min.

### (3) TGA

The TGA pattern of the crystal form is collected using METTLER TOLEDO TGA 1. The test temperature range is from 35° C. to 500° C., with a heating rate of 10° C./min. During the test, purging is performed with nitrogen at a flow rate of 50 mL/min.

### (4) DVS

The DVS pattern of the crystal form is tested using a DVS Intrinsic (SMS) at a test temperature of 25° C. and detected in cycle-DMDT mode.

### (5) Particle Size Analysis

The particle size data of the powder is analyzed using a Malvern Mastersizer 3000 laser particle sizer, and the particle size distribution of the crystal form A of revefenacin is determined with reference to Method 3 of General Chapter 0982, Chinese Pharmacopoeia, 2020 edition, Volume IV. Test method: The particle type is set as non-spherical, the particle refractive index is 1.52, the particle absorption rate is 0.001, and the rotation speed is 1800 rpm; the obscuration is 2%-15%, the sample background test time is 10 seconds, the dispersion solvent is water, the dispersant refractive index is 1.330, and the stabilization time is about 30 seconds; the number of tests is 3.

### (6) Analysis of Bulk Density, Tap Density and Angle of Repose

The bulk density and tap density of the crystal form A of revefenacin are determined using a powder density tester (equipment from Dandong Haoyu Technology Co., Ltd., model HY-100) with reference to the requirements of General Chapter 0993 Bulk Density and Tap Density, Chinese Pharmacopoeia, 2020 edition, Volume IV. Test method: An appropriate amount of the crystal form A sample is weighed, and slowly poured into a 50 mL glass graduated cylinder. The top surface is carefully leveled, and the apparent volume is recorded. The cylinder is then placed on the powder density tester and shaken at a frequency of 1250. The taped volume is recorded, and then the tap density is calculated. The same batch of samples is tested three times in parallel.

The angle of repose of the crystal form A of revefenacin is tested using a powder comprehensive characteristic tester (Dandong Bettersize Instruments Ltd., model BT-1000). Test method: An appropriate amount of the crystal form A sample is weighed and poured into a funnel. The powder falls through the funnel to form a cone, and then the angle between the inclined plane and the horizontal plane is determined. The test is repeated in triplicate.

### Example 1

90 g of revefenacin (which could be synthesized according to the method in Example 2 and Example 3 of patent application CN 102958916 A) and 360 mL of a mixed solvent of ethanol/water (volume ratio of ethanol:water being 1:1) were mixed and heated to 50° C. to give a clear solution. Then the solution was cooled to 45° C., and 0.1 g of seed crystals was then added. After crystal growth, 360 ml of a precipitation solvent (mixed solvent having a volume ratio of ethanol:water of 1:3) was slowly added dropwise. After completion of the dropwise addition, the mixture was cooled to 25° C. for crystal growth. Suction filtration was performed, and the filter cake was rinsed twice with 400 mL of a mixed solvent of ethanol/water (volume ratio of ethanol:water being 1:3). The resulting wet crystal product was finally dried at 30° C. and a vacuum degree of 0.1 Mpa for 20 h to obtain 82.3 g of the crystal form A of revefenacin, with an HPLC chemical purity of 99.9%, a yield of 91.4%, and a water content of 9.4 wt % as determined by Karl Fischer titration.

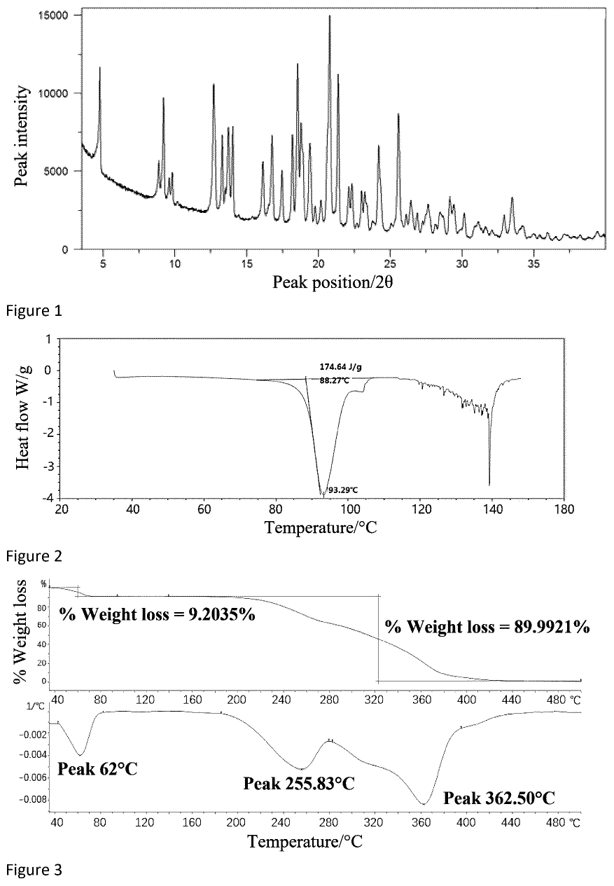

The XRPD pattern of the crystal form A of revefenacin is as shown in FIG. 1, and the XRPD peak positions and relative peak intensities are as shown in the following table:

| | Peak position | Relative peak |
|---|---|---|
| No. | 2θ (°) | intensity (%) |
| 1 | 4.7 | 46.4 |
| 2 | 8.9 | 17.7 |
| 3 | 9.2 | 49.9 |
| 4 | 9.6 | 10.7 |
| 5 | 9.8 | 14.8 |
| 6 | 10.2 | 1.7 |
| 7 | 12.7 | 87.7 |
| 8 | 13.3 | 38.0 |
| 9 | 13.7 | 46.5 |
| 10 | 14.0 | 42.1 |
| 11 | 14.4 | 1.3 |
| 12 | 15.4 | 1.7 |
| 13 | 16.1 | 31.4 |
| 14 | 16.7 | 42.7 |
| 15 | 17.4 | 26.1 |
| 16 | 18.2 | 46.8 |
| 17 | 18.5 | 81.4 |
| 18 | 18.8 | 45.2 |
| 19 | 19.4 | 40.5 |
| 20 | 19.8 | 9.2 |
| 21 | 20.1 | 11.3 |
| 22 | 20.7 | 100.0 |
| 23 | 21.3 | 80.8 |
| 24 | 22.1 | 20.9 |
| 25 | 22.3 | 23.4 |
| 26 | 22.7 | 3.5 |
| 27 | 23.0 | 21.0 |
| 28 | 23.3 | 16.2 |
| 29 | 23.8 | 4.3 |
| 30 | 24.2 | 40.4 |
| 31 | 25.0 | 3.3 |
| 32 | 25.6 | 68.1 |
| 33 | 26.1 | 9.5 |
| 34 | 26.4 | 14.7 |
| 35 | 26.9 | 10.1 |
| 36 | 27.2 | 6.1 |
| 37 | 27.6 | 13.0 |
| 38 | 28.1 | 4.4 |
| 39 | 28.5 | 11.9 |
| 40 | 29.1 | 18.6 |
| 41 | 29.4 | 16.3 |
| 42 | 30.1 | 13.8 |
| 43 | 30.8 | 3.2 |
| 44 | 31.1 | 6.5 |
| 45 | 31.6 | 5.5 |
| 46 | 32.1 | 3.6 |
| 47 | 32.9 | 12.1 |
| 48 | 33.5 | 22.6 |
| 49 | 34.1 | 5.7 |
| 50 | 34.9 | 2.3 |
| 51 | 35.4 | 2.6 |
| 52 | 35.9 | 4.1 |
| 53 | 36.5 | 2.3 |
| 54 | 37.2 | 2.8 |
| 55 | 38.2 | 2.3 |
| 56 | 38.8 | 0.3 |
| 57 | 39.4 | 3.3 |

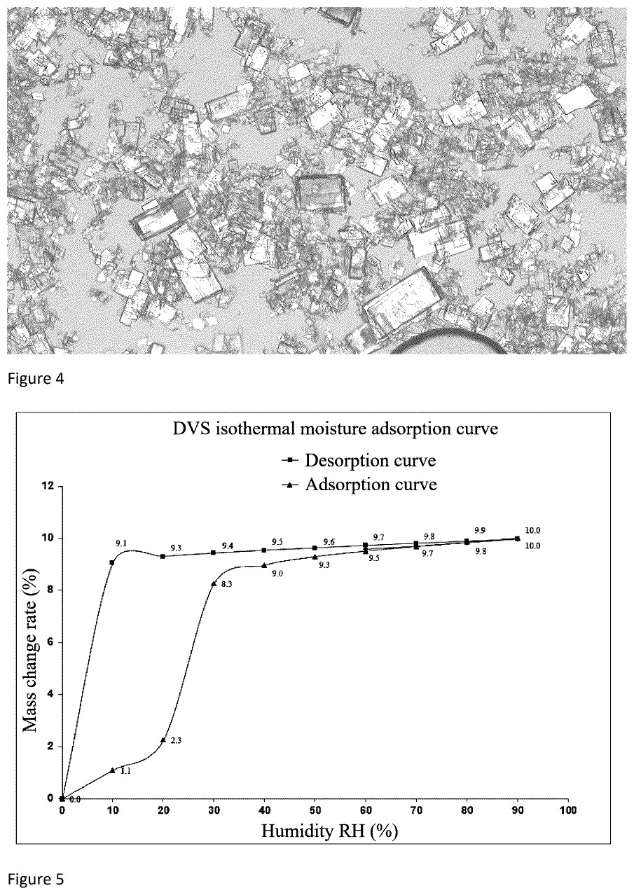

The thermal analysis DSC pattern of the crystal form A of revefenacin is as shown in FIG. 2, with an onset temperature of the endothermic peak at 88.3° C. The TGA of the crystal form A of revefenacin is as shown in FIG. 3, showing a weight loss of 9.2% due to the removal of water molecules of crystallization. The polarized light microscopy image of the crystal form A of revefenacin is as shown in FIG. 4.

### Example 1-1

2 g of revefenacin and 10 ml of a mixed solvent of acetone/water (volume ratio of acetone:water being 3:1) were mixed and heated to 55° C. to give a clear solution. Then the solution was cooled to 38° C., and 0.02 g of seed crystals was then added. After crystal growth, 8 ml of a precipitation solvent (mixed solvent having a volume ratio of acetone:water of 1:3) was slowly added dropwise. After completion of the dropwise addition, the mixture was cooled to 25° C. for crystal growth. Suction filtration was performed, and the filter cake was rinsed twice with 10 ml of a mixed solvent of acetone/water (volume ratio of acetone:water being 1:3). The resulting wet crystal product was dried at 25° C. and a vacuum degree of 0.1 Mpa for 10 h to obtain 1.85 g of the crystal form A of revefenacin, with a chemical purity of 99.79% and a yield of 92.5%.

The X-ray powder diffraction pattern, DSC pattern, TGA pattern and polarized light microscopy image of the resulting crystal form A of revefenacin are substantially identical to those of Example 1; and the water content as determined by Karl Fischer titration is 9.5 wt %.

The XRPD peak positions and relative peak intensities of the crystal form A of revefenacin are as shown in the following table:

| | Peak position | Relative peak |
|---|---|---|
| No. | 2θ (°) | intensity (%) |
| 1 | 4.8 | 46.6 |
| 2 | 8.9 | 23.8 |
| 3 | 9.3 | 64.2 |
| 4 | 9.7 | 10.5 |
| 5 | 9.9 | 16.9 |
| 6 | 10.3 | 1.7 |
| 7 | 12.8 | 76.9 |
| 8 | 13.4 | 43.7 |
| 9 | 13.8 | 52.5 |
| 10 | 14.1 | 43.8 |
| 11 | 14.5 | 1.1 |
| 12 | 15.5 | 1.1 |
| 13 | 16.2 | 28.6 |
| 14 | 16.9 | 42.5 |
| 15 | 17.5 | 23.4 |
| 16 | 18.2 | 45.0 |
| 17 | 18.5 | 68.5 |
| 18 | 18.8 | 64.4 |
| 19 | 19.5 | 42.1 |
| 20 | 19.8 | 7.2 |
| 21 | 20.2 | 11.7 |
| 22 | 20.8 | 100.0 |
| 23 | 21.4 | 67.5 |
| 24 | 22.2 | 19.7 |
| 25 | 22.4 | 19.5 |
| 26 | 23.1 | 16.4 |
| 27 | 23.4 | 18.6 |
| 28 | 24.3 | 34.7 |
| 29 | 25.3 | 3.5 |
| 30 | 25.7 | 55.0 |
| 31 | 26.1 | 6.4 |
| 32 | 26.5 | 13.9 |
| 33 | 27.0 | 7.8 |
| 34 | 27.7 | 17.4 |
| 35 | 28.6 | 10.5 |
| 36 | 29.2 | 13.6 |
| 37 | 29.5 | 16.5 |
| 38 | 30.2 | 10.7 |
| 39 | 31.2 | 7.3 |
| 40 | 31.8 | 3.7 |
| 41 | 32.2 | 2.6 |
| 42 | 33.0 | 8.6 |
| 43 | 33.6 | 21.6 |
| 44 | 34.3 | 5.6 |
| 45 | 35.1 | 2.1 |
| 46 | 35.5 | 1.2 |
| 47 | 36.1 | 2.9 |
| 48 | 36.7 | 1.0 |
| 49 | 37.3 | 2.3 |
| 50 | 38.0 | 1.0 |
| 51 | 38.3 | 1.2 |
| 52 | 39.5 | 2.6 |

### Example 2

2 g of revefenacin and 10 ml of a mixed solvent of ethanol/water (volume ratio of ethanol:water being 5:4) were mixed and heated to 55° C. to give a clear solution. Then the solution was cooled to 44° C., and 0.02 g of seed crystals was then added. After crystal growth, 8 mL of a precipitation solvent (mixed solvent having a volume ratio of ethanol:water of 1:5) was slowly added dropwise. After completion of the dropwise addition, the mixture was cooled to 25° C. and subjected to crystal growth. Suction filtration was performed, and the filter cake was rinsed twice with 10 ml of a mixed solvent of ethanol/water (volume ratio of ethanol:water being 1:5). The resulting wet crystal product was dried at 25° C. and a vacuum degree of 0.1 Mpa for 17 h to obtain 1.88 g of the crystal form A of revefenacin, with a chemical purity of 99.79% and a yield of 94%. The X-ray powder diffraction pattern, DSC pattern, TGA pattern and polarized light microscopy image of the resulting crystal form A of revefenacin are substantially identical to those of Example 1; and the water content as determined by Karl Fischer titration is 9.1 wt %.

### Example 3

10 g of revefenacin and 40 mL of a mixed solvent of ethanol/water (volume ratio of ethanol:water being 2:1) were mixed and heated to 52° C. to give a clear solution. Then the solution was cooled to 35° C., and 0.2 g of seed crystals was then added. After crystal growth, 60 ml of a precipitation solvent (mixed solvent having a volume ratio of ethanol:water of 1:4) was slowly added dropwise. After completion of the dropwise addition, the mixture was cooled to 25° C. for crystal growth. Suction filtration was performed, and the filter cake was rinsed twice with 60 ml of a mixed solvent of ethanol/water (volume ratio of ethanol:water being 1:4). The resulting wet crystal product was dried at 25° C. and a vacuum degree of 0.1 Mpa for 2 h to obtain 9.30 g of the crystal form A of revefenacin, with a chemical purity of 99.87%, a yield of 92.6%, and a water content of 9.3 wt % as determined by Karl Fischer titration. The X-ray powder diffraction pattern, DSC pattern, TGA pattern and polarized light microscopy image of the resulting crystal form A of revefenacin are substantially identical to those of Example 1.

### Example 4

An excess amount of revefenacin was added to an acetone/water (volume ratio being 6:1) solution, mixed uniformly, and then filtered via a 0.25 μm syringe filter to obtain a saturated solution of revefenacin. 0.5 mL of the saturated solution of revefenacin was taken, mixed with 4.5 mL of an acetone/water solution for dilution, and left to stand for very slow evaporation to obtain a revefenacin hydrate single crystal.

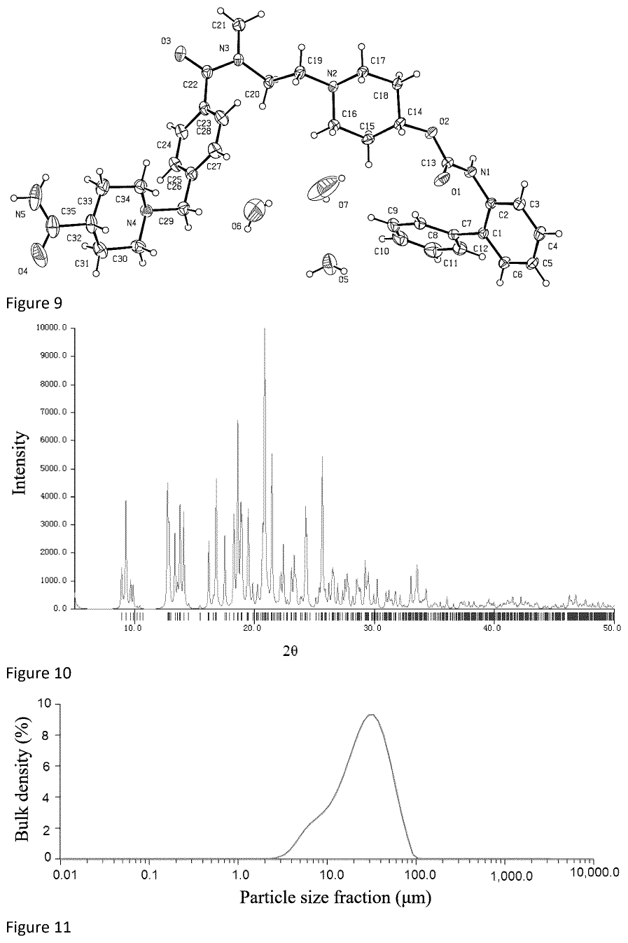

The single crystal structure diagram of the revefenacin hydrate single crystal is as shown in FIG. 9, and the simulated XRD pattern calculated from the single crystal is as shown in FIG. 10.

### Test Example 1: Dynamic Vapor Sorption Evaluation

Dynamic vapor sorption curve (also referred to as adsorption-desorption characteristic curve) was evaluated using a DVS Intrinsic (SMS). Approximately 30 mg of the sample prepared in Example 1 was subjected to a three-stage programmed scanning method at room temperature (25° C.) as follows: the first-stage hygroscopicity program involving a stepwise increase in relative humidity from 60% RH to 90% RH, with a relative humidity change rate of 10% RH/step; the second-stage hygroscopicity program involving a stepwise decrease in relative humidity from 90% RH to 0% RH, with a relative humidity change rate of 10% RH/step; and the third-stage hygroscopicity program involving a stepwise increase in relative humidity from 0% RH to 90% RH, with a relative humidity change rate of 10% RH/step. The mass change rate mode (dm/dt mode) was used, where when the mass change rate (dm/dt) was ≤0.005%, the instrument program controlled the switching of the relative humidity (RH) to the next stage and maintained constant humidity (+/−10% RH).

The DVS (dynamic vapor sorption) curve of the crystal form A of revefenacin of Example 1 is as shown in FIG. 5, showing reversible adsorption/desorption characteristics and low hygroscopicity. During the process of gradually increasing the humidity from 60% RH to 90% RH, the hygroscopic weight gain of the sample is <0.4%; within the range of 30%-90% RH, the hygroscopic weight gain of the samples is <2%, and the crystal form of the samples collected during the process remains unchanged.

### Test Example 2

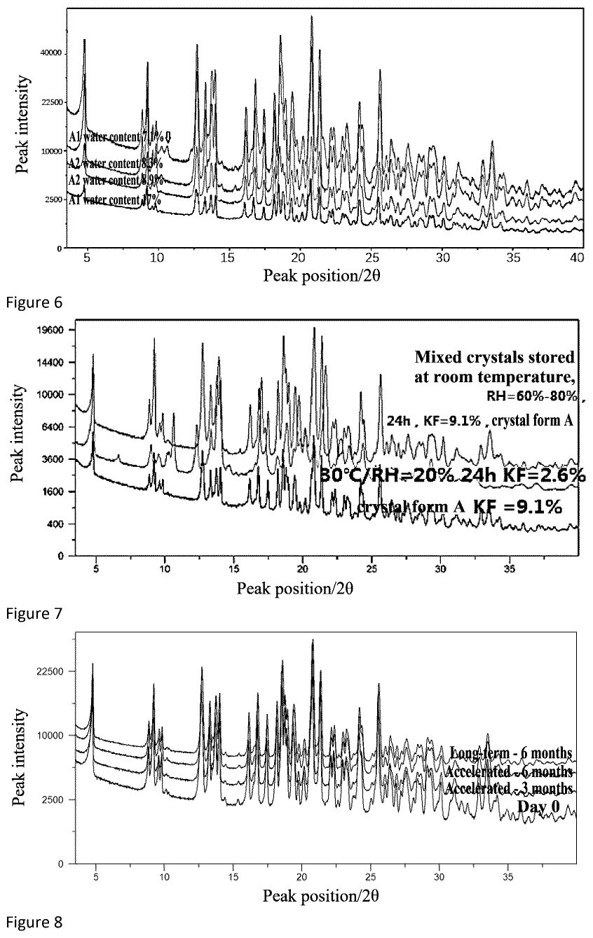

The wet crystal product of Example 1 was dried under conditions of a temperature of 30° C.-35° C. and a vacuum degree of −0.08 Mpa. During the drying, the samples were collected for Karl Fischer water content testing and XRPD. The results showed that when KF water content=17%, KF water content=8.9%, and KF water content=8.3%, the crystal forms were all crystal form A; when the water content continued to decrease to KF water content=7.1%, “impurity peaks” began to appear in the XRPD pattern, as shown in FIG. 6, indicating that a crystal form transformation occurred when KF water content=7.1%, with mixed crystals formed.

### Test Example 3

The crystal form A of revefenacin of Example 2 was dried in an oven at a temperature of 30° C., a vacuum degree of 0.098 Mpa to 0.1 Mpa, and a humidity of ≤20% for 24 h, labeled as sample 1; and sample 1 was then stored under conditions of an ambient humidity RH of 60%-80% and a temperature of 25° C.-30° C. for 24 h, labeled as sample 2. Samples 1 and 2 were tested for XRPD, HPLC purity, and KF water content. The results are as shown in the table below and in FIG. 7.

| | | | KF/water |
|---|---|---|---|
| Sample | XRPD | HPLC % | content |
| Crystal form of | Crystal form A | 99.79% | 9.1% |
| Example 2 | | | |
| Sample 1 | Mixed crystals | 99.72% | 2.6% |
| Sample 2 | Crystal form A | 99.76% | 9.1% |

Based on the analysis of FIG. 7 and the above table, it can be seen that after dehydration of the crystal form A of revefenacin, mixed crystals appear, and when the humidity is changed, the mixed crystals adsorb environmental moisture and transform back into crystal form A; and the chemical purity of the crystal form remains unchanged during the temperature- and humidity-mediated mutual transformation of crystal forms.

### Test Example 4

4.1 The crystal form A of revefenacin prepared in Example 3 was packaged in packaging materials (an inner packaging of two low-density polyethylene medicinal bags, plus one polyester/aluminum/polyethylene composite bag compliant with pharmaceutical packaging standards). The crystal form A packaged in the packaging materials was subjected to the following conditions for investigating influencing factors: high temperature (40° C.), high humidity (92.5% RH), illumination (4500 lx illumination; near ultraviolet ≥0.8×102 ρW/cm²) and accelerated conditions (40° C./75% RH). Samples were collected and tested for XRPD and HPLC purity at day 0, day 5, day 10, and day 35, respectively. The results are as shown in the table below.

| | Time | | | |
|---|---|---|---|---|
| Condition | day 0 | day 5 | day 10 | day 35 |
| 40° C. | Purity: 99.87% | Crystal | Crystal | 99.87% |
| | XRPD: Crystal | form A | form A | Crystal form A |
| 92.5% RH | form A | Crystal | Crystal | 99.91% |
| (25° C.) | | form A | form A | Crystal form A |
| Illumination | | Crystal | Crystal | 99.89% |
| at 25° C. | | form A | form A | Crystal form A |
| 40° C./75% RH | | Crystal | Crystal | 99.94% |
| | | form A | form A | Crystal form A |

Based on the above table, it can be seen that the crystal form A of revefenacin of the present disclosure remains unchanged when exposed to high temperature (40° C.), high humidity (92.5% RH), illumination (4500 lx illumination; near ultraviolet ≥0.8×102 μW/cm²) and accelerated (40° C./75% RH) conditions, showing good stability.

4.2 The crystal form A of revefenacin prepared in Example 3 was packaged in packaging materials (an inner packaging of two low-density polyethylene medicinal bags, plus one polyester/aluminum/polyethylene composite bag compliant with pharmaceutical packaging standards). The packaged samples were stored under conditions of 30° C./35% RH and 40° C./75% RH, and collected and investigated for crystal forms at day 0, 3 months, and 6 months, respectively. The results are as shown in the table below and in FIG. 8.

| Condition | day 0 | 3 months | 6 months |
|---|---|---|---|
| 40° C./75% RH | Crystal form A | Crystal form A | Crystal form A |
| 30° C./35% RH | Crystal form A | / | Crystal form A |

Based on the above table and FIG. 8, it can be seen that the revefenacin of the present disclosure remains unchanged in both crystal form and crystallinity during the 3-month storage and 6-month storage under the accelerated condition (40° C./75% RH); and after stored under long-term conditions (30° C./35% RH) for 6 months, the crystal form A remains unchanged in both crystal form and crystallinity.

### Test Example 5

An appropriate amount of the crystal form A prepared in Example 1 was taken, and the powder properties thereof were evaluated. The particle size distribution of the samples was investigated using a Malvern 3000 particle size analyzer; the angle of repose of the crystal form A was tested using a powder comprehensive characteristic tester; and the bulk density and tap density of the crystal form A were tested using a powder density tester.

The particle size distribution curve of the crystal form A is as shown in FIG. 11. The crystal form A of the present disclosure shows a unimodal particle size distribution curve with uniform particle size distribution, showing larger primary particle size: D10=8.40 μm; D50=25.86 μm; D90=53.59 μm.

The crystal form A of the present disclosure has superior fluidity, with a powder angle of repose of 45°, and both the bulk density (0.285 g/cm³) and tap density (0.494 g/cm³) are relatively high. The superior powder properties are convenient for pharmaceutical processing operations, such as subpackaging and mixing of materials during the preparation process.

### Test Example 6

The crystal form A of revefenacin of Example 1 and Example 3 were respectively dissolved in citric acid/sodium citrate buffered physiological saline, and the pH was controlled to 5.0±0.5 to prepare revefenacin solutions, labeled as revefenacin solution 1 # and 2 #, respectively. After revefenacin solution 1 # and 2 # and commercial samples were stored under accelerated conditions (40° C./75% RH) for one month and long-term conditions (25° C.±2° C./40%±5% RH) for one month or three months, less than 0.2% degradation was observed in all samples, with total impurity content of less than 0.4%.

| | % | % |
|---|---|---|
| | Total | Increase |
| | impu- | in total |
| Sample name | rity | impurity |
| Revefenacin solution 1# - day 0 | 0.06 | / |
| Revefenacin solution 2# - day 0 | 0.22 | / |
| Commercially available sample day - 0 | 0.45 | / |
| Revefenacin solution 2# - accelerated 1 month | 0.34 | 0.12 |
| Commercially available sample - accelerated 1 month | 0.62 | 0.17 |
| Revefenacin solution 1# - long-term 1 month | 0.11 | 0.05 |
| Revefenacin solution 1# - long-term 2 months | 0.12 | 0.06 |
| Revefenacin solution 1# - long-term 3 months | 0.16 | 0.10 |
| Revefenacin solution 2# - long-term 1 month | 0.24 | 0.02 |
| Revefenacin solution 2# - long-term 3 months | 0.24 | 0.02 |

Note:
The commercially available sample was revefenacin inhalation solution under tradename YUPELRI; the increase in total impurity was the amount of increase compared to the 0-day total impurity.

The crystal form A of revefenacin and the method for preparing same disclosed in the present application can be achieved by those skilled in the art by making appropriate changes to raw materials, process parameters, etc. in view of the content herein. The method and product of the present application have been described by way of preferred examples, and relevant those skilled in the art can obviously make changes or appropriate modifications and combinations of the method and product described herein without departing from the content, spirit and scope of the present disclosure, so as to realize the technology of the present application. It should be particularly pointed out that all similar substitutions and modifications are obvious to those skilled in the art, and are deemed to be included in the spirit, scope and content of the present application.

1. A crystal form A of revefenacin, wherein the crystal form A of revefenacin comprises an X-ray powder diffraction pattern having characteristic peaks at diffraction angles 2θ of 4.7±0.2°, 9.2±0.2°, 12.7±0.2°, 18.2±0.2°, 18.5±0.2°, 20.7±0.2°, 21.3±0.2° and 25.6±0.2°.

2. The crystal form A of revefenacin according to claim 1, wherein the crystal form A of revefenacin comprises an X-ray powder diffraction pattern substantially as shown in FIG. 1.

3. The crystal form A of revefenacin according to claim 1, wherein the crystal form A of revefenacin has one or more of the following characteristics:
1. the crystal form A of revefenacin is a hydrate crystal form containing water of crystallization and having a water content of 8.0 wt %-10 wt % as determined by Karl Fischer titration;
2. the crystal form A of revefenacin has an endothermic peak between 85° C. and 125° C., and the endothermic peak has an onset temperature of 88.3° C.±5° C.;
3. the crystal form A of revefenacin comprises a DSC curve substantially as shown in FIG. 2;
4. the crystal form A of revefenacin shows a weight loss of 9.2%±0.2% in a temperature range of 35° C.-100° C.;
5. the crystal form A of revefenacin comprises a TGA curve substantially as shown in FIG. 3;
6. the crystal form A of revefenacin features a regular sheet shape; and/or
7) the crystal form A of revefenacin is a revefenacin trihydrate.

4. A revefenacin hydrate single crystal, wherein the single crystal belongs to the triclinic crystal system and has a space group of P-1, with crystal axes a=9.5522 (4) Å, b=9.9096 (4) Å, and c=18.3690 (9) Å, interaxial angles α=86.419 (4)°, β=86.906 (4)°, and γ=89.544 (3)°, a Z value of 2 for the number of asymmetric units per unit cell, and each unit cell containing one revefenacin molecule and three water molecules.

5. A method for preparing the crystal form A of revefenacin according to claim 1, wherein the preparation method comprises the following steps:
step I: mixing revefenacin with a dissolution solvent, stirring and heating the resulting mixture to give a clear solution, wherein the dissolution solvent is a mixed solvent of ethanol/water or a mixed solvent of acetone/water;
step II: cooling the solution to 35° C.-45° C., stirring the solution while maintaining the temperature for crystal growth to obtain a revefenacin suspension solution;
step III: under a condition of 35° C.-45° C., adding a precipitation solvent dropwise to the suspension solution obtained in step II; and
step IV: cooling the suspension solution to 0° C.-25° C. for crystal growth, and then performing filtration and drying to a constant weight to obtain the crystal form A of revefenacin.

6. The method for preparing the crystal form A of revefenacin according to claim 5, wherein the preparation method comprises one or more of the following characteristics:
I) in step I, the mass-to-volume ratio of revefenacin to the dissolution solvent, expressed in g/mL, is 1:4-5;
II) in step I, revefenacin is added to the dissolution solvent, stirred, and heated to 50° C.-55° C. to give a clear solution;
III) the dissolution solvent is a mixed solvent of ethanol/water or the dissolution solvent is a mixed solvent of acetone/water;
IV) in step II, seed crystals of the crystal form A of revefenacin are added;
V) in step II, the seed crystals are added in an amount of 1 wt % to 2 wt % the revefenacin;
VI) in step II, stirring while maintaining the temperature for crystal growth is performed over 1-4 h; the stirring rate is 100-800 rpm;
VII) in step III, the precipitation solvent is a mixed solvent of ethanol and water or a mixed solvent of acetone/water, such as a mixed solvent of ethanol and water in a volume ratio of 1:3-5 or a mixed solvent of acetone and water in a volume ratio of 1:3-5;
VIII) the mass-to-volume ratio of revefenacin to the precipitation solvent in step III, expressed in g/mL, is 1:3-6;
IX) in step III, the dropwise addition rate of the precipitation solvent is non-linear dropwise addition, with the dropwise addition rate being slow first and then fast;
X) in step IV, the crystal growth is maintained for 1-10 h;
XI) in step IV, the cooling rate is 0.1° C./min-2° C./min; and/or
XII) in step IV, drying is performed at a temperature of 25° C.-45° C. and a vacuum degree of 0.08 Mpa-0.1 Mpa for 2-20 h.

7. A method for preparing the revefenacin hydrate single crystal according to claim 4, wherein the preparation method comprises the following steps:
step I: adding an excess amount of revefenacin to an acetone/water solution, mixing uniformly, and filtering the resulting mixture to obtain a saturated solution of revefenacin; and
step II: diluting the saturated solution of revefenacin with an acetone/water solution, and leaving the solution to stand for evaporation to obtain the revefenacin hydrate single crystal.

8. The method for preparing the revefenacin hydrate single crystal according to claim 7, wherein the preparation method comprises one or more of the following characteristics:
i) in steps I and II, the volume ratio of acetone to water is 5-7:1; and/or
ii) in step I, filtering is performed via a syringe filter; and/or
iii) in step II, the volume ratio of the saturated solution of revefenacin to the acetone/water solution is 1:8-10.

9. A pharmaceutical composition, comprising the crystal form A of revefenacin according to claim 1 and one or more pharmaceutically acceptable carriers.

10. The pharmaceutical composition according to claim 9, comprising one or more other therapeutic agents, wherein the other therapeutic agents refer to pharmacologically active substances other than revefenacin.

11. (canceled)

12. The crystal form A of revefenacin according to claim 1, wherein the crystal form A of revefenacin comprises an X-ray powder diffraction pattern having characteristic peaks at diffraction angles 2θ of 4.7±0.2°, 9.2±0.2°, 12.7±0.2°, 13.3±0.2°, 13.7±0.2°, 14.0±0.2°, 16.1±0.2°, 16.7±0.2°, 17.4±0.2°, 18.2±0.2°, 18.5±0.2°, 18.8±0.2°, 19.4±0.2°, 20.7±0.2°, 21.3±0.2°, 22.1±0.2°, 22.3±0.2°, 23.0±0.2°, 24.2±0.2°, 25.6±0.2° and 33.5±0.2°.

13. The crystal form A of revefenacin according to claim 1, wherein the crystal form A of revefenacin comprises an X-ray powder diffraction pattern having characteristic peaks at diffraction angles 2θ of 4.7±0.2°, 8.9±0.2°, 9.2±0.2°, 9.6±0.2°, 9.8±0.2°, 12.7±0.2°, 13.3±0.2°, 13.7±0.2°, 14.0±0.2°, 16.1±0.2°, 16.7±0.2°, 17.4±0.2°, 18.2±0.2°, 18.5±0.2°, 18.8±0.2°, 19.4±0.2°, 20.1±0.2°, 20.7±0.2°, 21.3±0.2°, 22.1±0.2°, 22.3±0.2°, 23.0±0.2°, 23.3±0.2°, 24.2±0.2°, 25.6±0.2°, 26.4±0.2°, 26.9±0.2°, 27.6±0.2°, 28.5±0.2°, 29.1±0.2°, 29.4±0.2°, 30.1±0.2°, 32.9±0.2° and 33.5±0.2°.

14. The crystal form A of revefenacin according to claim 1, wherein the crystal form A of revefenacin comprises a polarized light microscopy (PLM) image as shown in FIG. 4.

15. The method for preparing the crystal form A of revefenacin according to claim 6, wherein the dissolution solvent is a mixed solvent of ethanol and water in a volume ratio of 1-2:1, or the dissolution solvent is a mixed solvent of acetone and water in a volume ratio of 1-4:1.

16. The method for preparing the crystal form A of revefenacin according to claim 6, wherein the dropwise addition is performed over 2-6 h.

17. The pharmaceutical composition according to claim 10, wherein the other therapeutic agents comprise other muscarine receptor antagonists or anticholinergic activity capable of exerting synergistic therapeutic effects with revefenacin.

18. A method for treating chronic obstructive pulmonary disease, comprising administering to a subject in need thereof a therapeutically effective amount of the crystal form A of revefenacin according to claim 1.
